(19) United States
(12) Patent Application Publication
(10) Pub. No.: US 2023/0288398 A1
(43) Pub. Date: Sep. 14, 2023

(54) Systems and Methods for Gestational Age Dating and Applications Thereof

(71) Applicant: The Board of Trustees of the Leland Stanford Junior University, Stanford, CA (US)

(72) Inventors: Kevin Contrepois, Brisbane, CA (US); Michael P. Snyder, Stanford, CA (US); Songjie Chen, Newark, CA (US); Nima Aghaeepour, Palo Alto, CA (US); Mohammad Sajjad Ghaemi, Stanford, CA (US)

(73) Assignee: The Board of Trustees of the Leland Stanford Junior University, Stanford, CA (US)

(21) Appl. No.: 18/251,703

(22) PCT Filed: Nov. 8, 2021
(86) PCT No.: PCT/US2021/072296

§ 371 Date: May 3, 2023

Related U.S. Application Data
(60) Provisional application No. 63/110,868, filed on Nov. 6, 2020.

(51) Int. Cl. G01N 33/493; G16H 50/20

(52) U.S. Cl. CPC G01N 33/493; G16H 50/20

(57) ABSTRACT

Methods to compute gestational age and applications thereof are described. Generally, systems utilize analyte measurements derived from a urine sample to determine a gestational age, which can be used as a basis to perform interventions and treat individuals.

### CROSS REFERENCE TO RELATED APPLICATIONS

This application claims priority to U.S. Provisional Application Ser. No. 63/110,868, entitled “Methods for Gestational Age Dating and Applications Thereof,” filed Nov. 6, 2020, which is incorporated herein by reference in its entirety.

### FIELD OF TECHNOLOGY

The disclosure is generally directed to processes to follow pregnancy progression by defining a metabolic clock of pregnancy and detect early adverse outcomes of pregnancy (e.g. intrauterine growth restriction, preterm birth, preeclampsia) and applications thereof, and more specifically to methods that estimate gestational age (GA) and time to labor using metabolite levels.

### BACKGROUND

Pregnancy is one of the most critical periods for mother and child. It involves a tremendous flow of physiological changes and metabolic adaptations week by week, and even small deviations from the norm may have detrimental consequences. There are 300,000 pregnancy and birth-related maternal deaths and 7.5 million perinatal deaths annually worldwide. In addition, 30% of all pregnancies end in miscarriage (<20 weeks), and preterm birth (<37 weeks). The latter is the leading cause of global neonatal morbidity and mortality and is observed for 7-17% of all pregnancies. With 170 million pregnancies yearly worldwide, even small improvements in obstetric health care, based on a better understanding of how pregnancy is regulated, may impact on the wellbeing of a large number of women and children.

Assessment of gestational age is key to provide optimal care during pregnancy. However, its accurate determination remains challenging in low- and middle-resource countries, where access to obstetric ultrasound is limited. Hence, there is an urgent need to develop clinical approaches that allow accurate and inexpensive estimations of GA.

### SUMMARY

Various embodiments are directed towards systems and methods for assessing gestational age. In various embodiments, a trained computational model utilizes measurements of metabolites derived from a pregnant individual to determine gestational progress. In various embodiments, the computational model is trained utilizing analyte measurements derived from urine samples of a cohort of pregnant individuals. In various embodiments, to determine gestational age, metabolites are collected from the pregnant individual at one or more timepoints and measured. In various embodiments, the measurements of collected metabolites are utilized within the trained computational model to determine gestational age.

In an embodiment, a gestational age of a pregnant individual is determined. One or more analytes of a urine sample collected from an individual is measured. Using a predictive computational model and the one or more analyte measurements, a gestational age of the individual is estimated.

### BRIEF DESCRIPTION OF THE DRAWINGS

The description and claims will be more fully understood with reference to the following figures and data graphs, which are presented as exemplary embodiments of the invention and should not be construed as a complete recitation of the scope of the invention.

FIG. 1 provides a flow chart of a method for determining gestational age in accordance with various embodiments.

FIG. 2 provides a flow chart of a method to construct and train a computational model to determine a pregnant individual’s gestational age or gestational in accordance with various embodiments.

FIG. 3 provides a flow chart of a method for determining gestational age or gestational health using a computational model in accordance with various embodiments.

FIG. 4 provides a schematic of a study design for analyzing urine samples collected from pregnant women across five sites and analyzed using broad-spectrum metabolomics LC-MS in accordance with various embodiments.

FIG. 5 provides data graphs depicting gestational age at collection of urine and gestational age at delivery, utilized in accordance with various embodiments.

FIG. 6 provides a data table with demographics and birth certificates of the cohort utilized to generate a computational model to predict gestational age, utilized in accordance with various embodiments.

FIG. 7 provides a pie chart depicting structural categorization of detected urine metabolites according to the “Superclass level” of the ClassyFire classification system, utilized in accordance with various embodiments.

FIG. 8 provides principal component analysis plots of data generated in HILIC and RPLC modes, generated in accordance with various embodiments. The study samples were intermixed suggesting limited batch effect and the QCs clustered together indicating good technical reproducibility. Each dot represents a sample colored by batch information.

FIG. 9 provides hierarchical clustering of all the samples analyzed in the study (n = 172), generated in accordance with various embodiments. Clustering distance = Spearman, clustering method = complete. Multiple aliquots for each sample were processed and analyzed in a random order. Branches in red indicate duplicate samples that present a tight clustering demonstrating the quality of the assay. The mean of duplicate samples was used for downstream analysis.

FIG. 10 provides mass spectrometry intensity plots depicting dilution effect correction using probabilistic quotient normalization (PQN), generated in accordance with various embodiments. The distribution of MS signal intensity was variable across samples and became comparable after normalization.

FIG. 11 provides principal component analysis of all the samples in the study (n = 99), generated in accordance with various embodiments. The study samples were mainly intermixed suggesting limited sample collection and handling variability across sites.

FIGS. 12 and 13 provide data plots of random forest (RF) modeling of GA at sampling (FIG. 12) and at delivery (FIG. 13) using all the samples in the study (n = 99), generated in accordance with various embodiments. The shaded area represents the 95% confidence interval.

FIG. 14 provides data plots depicting performance of the restricted RF prediction model ofGA that uses three metabolites (C19H26O7S, C24H30O9 and estriol glucuronide) and all the samples in the study (n = 99), generated in accordance with various embodiments. The model was validated in an independent cohort (n = 20). The blue area represents the 95% confidence interval.

FIG. 15 provides principal component analysis using predictive metabolites (P-value < 0.05), generated in accordance with various embodiments. PC1 and PC4 were chosen because they associated the most strongly with GA.

FIG. 16 provides Kegg metabolic enrichment analysis, generated in accordance with various embodiments.

FIG. 17 provides a volcano plot of annotated significant metabolites (P-value < 0.05), generated in accordance with various embodiments. Beta coefficients were calculated using a linear modeling and P-values were calculated from Spearman correlations.

FIG. 18 provides data graphs of the top 6 metabolites in the predictive model and LOESS fit across all the samples, generated in accordance with various embodiments. The shaded area represents the 95% confidence interval.

FIG. 19 provides a data graph depicting distribution of GA at sample collection in term (n = 49) and preterm pregnancies (n = 50), generated in accordance with various embodiments.

FIG. 20 provides data graphs depicting performance of the RF prediction models of GA in term and preterm deliveries, generated in accordance with various embodiments.

FIG. 21 provides a dot plot of p-values of selected metabolites in term and preterm RF models, generated in accordance with various embodiments. Metabolites that are most predictive tend to be significant in both models.

FIG. 22 provides a data graph depicting coefficient of variation of the top 10 metabolites across GA ranges, generated in accordance with various embodiments.

FIG. 23 provides data graphs depicting top 10 metabolites in both term and preterm predictive models and LOESS fit, generated in accordance with various embodiments. The shaded areas represent the 95% confidence interval.

FIG. 24 provides a data graph depicting number of samples and origin of samples across gestational age, generated in accordance with various embodiments.

FIG. 25 provides a dot plot depicting importance of metabolites in term and preterm RF models in accordance with various embodiments.

### DETAILED DESCRIPTION

Turning now to the drawings and data, methods to determine gestational age based on analyte measurements derived from a pregnant individual and applications thereof in accordance with various embodiments are described. In some embodiments, analytes are derived from a urine sample of a pregnant individual. In some embodiments, a panel of analyte measurements are used to compute gestational age and provide an indication of an individual’s pregnancy timeline. Many embodiments utilize an individual’s gestational age and/or health determination to perform further diagnostic testing and/or treat the individual. In some instances, a diagnostic can include periodic medical checkups, fetal monitoring, blood tests (e.g., glucose), microbial culture tests, genetic screening, chorionic villus sampling, and amniocentesis. In some instances, a treatment can include a medication, a dietary supplement, caesarian delivery, a surgical procedure, and any combination thereof.

Many treatment regimens and clinical decisions in obstetrics depend on an accurate estimation of the timing and progression of pregnancy. Current clinical determination of gestational age and due date are typically based on information about last menstruation date or ultrasound imaging, which can be imprecise or unavailable in various regions of the world. An accurate and cost-effective method for estimating gestational age and delivery time is in need.

The present disclosure is based on the discovery of analyte biomarkers that can be used in monitoring women during pregnancy to determine gestational age and time until delivery. Untargeted analyte investigations were performed on urine samples collected from pregnant women from diverse locations of the world (see Exemplary Embodiments). This study revealed analytes derived from urine that can estimate gestational age. Many analyte measurements and the dynamics of the various analytes were shown to be timed precisely according to pregnancy progression and can be used to assess gestational progress. In various embodiments, computational models utilize analyte measurements to determine gestational age and health status.

### Analytes Indicative of Pregnancy Progression and Health Status

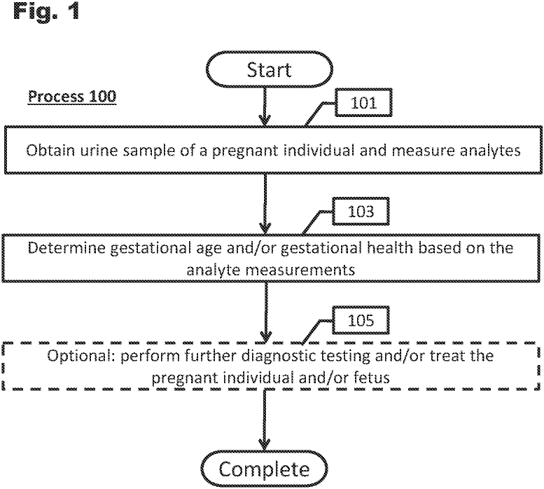

A process for determining gestational age using analyte measurements, in accordance with an embodiment of the disclosure is shown in FIG. 1. This embodiment is directed to determining gestational age, track pregnancy progression and inform health status of an individual.

In some embodiments, metabolites are to include intermediates and products of metabolism such as (for example) sugars, amino acids, nucleotides, antioxidants, organic acids, polyols, vitamins, and the like. In various embodiments, protein constituents are chains of amino acids which are to include (but not limited to) organic acids, organoheterocyclic compounds, lipids and lipid-like molecules, benzenoids, organic oxygen compounds and other minor chemical classes. In some embodiments, lipids and lipid-like molecules are a broad class of molecules that include (but are not limited to) sterols (e.g.., steroid hormones), fatty acid molecules, fat soluble vitamins, glycerolipids, phospholipids, sphingolipids, prenols, saccharolipids, polyketides, and the like.

In some embodiments, clinical data and/or personal data can be additionally used to indicate gestation age. In some embodiments, clinical data is to include medical patient data such as (for example) weight, height, heart rate, blood pressure, body mass index (BMI), clinical tests, medication regimen and the like.

Referring back to FIG. 1, process 100 begins with obtaining one or more biological specimens and measuring (101) analytes from a pregnant individual. In many instances, analytes are measured from a urine sample. In some embodiments, an individual’s sample is collected during fasting, or in a controlled clinical assessment. In some embodiments, a single urine measurement is collected. In some embodiments, analytes are collected over a period a time (e.g., across pregnancy timeline) and measured at each time point, resulting in a dynamic analysis of the analytes. In some of these embodiments, analytes are measured with periodicity (e.g., weekly, monthly, trimester).

In a number of embodiments, an individual is any individual that has their analytes collected and measured, especially individuals that have an indication of pregnancy. In some embodiments, an individual has been diagnosed as being pregnant (e.g., as determined by urine, blood test or ultrasound). Embodiments are also directed to an individual being one that has not yet been diagnosed as pregnant.

A number of analytes can be used to indicate gestation age, including (but not limited to) organic acids, organoheterocyclic compounds, lipids and lipid-like molecules, benzenoids, organic oxygen compounds and other minor chemical classes. In some embodiments, clinical data and/or personal data can be additionally used to indicate gestation age and/or health. Analytes can be detected and measured by a number of methods, including nucleic acid and protein sequencing, mass spectrometry, colorimetric analysis, immunodetection, and the like.

In several embodiments, analyte measurements are performed by taking a single time-point measurement. In many embodiments, the median and/or average of a number of time points for participants with multiple time-point measurements are utilized. Various embodiments incorporate correlations, which can be calculated by a number of methods, such as the Spearman correlation method. A number of embodiments utilize a computational model that incorporates analyte measurements, such as linear regression, random forest regression, and elastic net models. Significance can be determined by calculating p-values and/or contribution, which may be corrected for multiple hypotheses testing. It should be noted however, that there are several correlation, computational models, and statistical methods that can utilize analyte measurements and may also fall within some embodiments of the invention.

In a number of embodiments, dynamic correlations use a ratio of analyte measurements between two time points, a percent change of analyte measurements over a period of time, a rate of change of analyte measurements over a period of time, or any combination thereof. Several other dynamic measurements may also be used in the alternative or in combination in accordance with multiple embodiments.

Using static and/or dynamic measures of analytes, process 100 determines (103) gestational age and/or gestational health based on the analyte measurements. In many embodiments, the correlations and/or computational models can be used to indicate gestational age pregnancy progression and health status. In some embodiments, GA is determined prior to week 13, at which point fetus size is the gold standard to determine GA. In some embodiments, gestational age is predicted between weeks 8 and 19. In several embodiments, determining analyte correlations or modeling gestational age is used to substitute other gestational tests, such as (for example) ultrasonography. In various embodiments, measurement of analytes can be used as a precursor indicator to determine whether to perform a further clinical test, such as (for example) ultrasonography.

Having determined an individual’s gestational age status, further diagnostic test can optionally be performed or the pregnant individual and/or fetus can be treated (105). In some instances, a diagnostic can include periodic medical checkups, fetal monitoring, blood tests (e.g., glucose), microbial culture tests, genetic screening, chorionic villus sampling, amniocentesis, and any combination thereof. In some instances, a treatment can include a medication, a dietary supplement, caesarian delivery, a surgical procedure, and any combination thereof.

While specific examples of determining GA or gestational health are described above, one of ordinary skill in the art can appreciate that various steps of the process can be performed in different orders and that certain steps may be optional according to some embodiments of the invention. As such, it should be clear that the various steps of the process could be used as appropriate to the requirements of specific applications. Furthermore, any of a variety of processes for determining GA or gestational health are appropriate to the requirements of a given application can be utilized in accordance with various embodiments of the invention.

### Modeling Pregnancy Progression and Health With Analyte Measurements

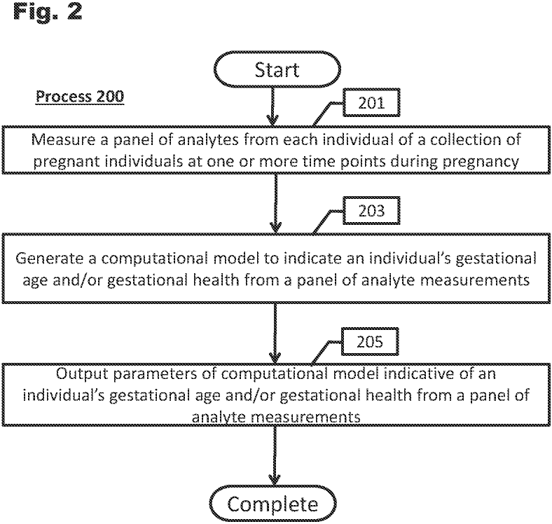

A process for constructing and training a computational model to estimate GA or gestational health in accordance with various embodiments is shown in FIG. 2. Process 200 measures (201) a panel of analytes from each individual of a collection of pregnant individuals at a single time during pregnancy. In several embodiments, analytes are measured from a urine sample of an individual. In some embodiments, an individual’s sample is collected during fasting. A number of methods are known to collect samples from an individual and can be used within various embodiments of the invention. In several embodiments, analytes are collected and measured at a single or multiple time points, resulting in a static or a dynamic analysis of the analytes.

In several embodiments, analytes are collected with periodicity across the timeline of pregnancy. In some embodiments, analytes are collected prior to week 13 of gestation. In some embodiments, analytes are collected between weeks 8 and 19 of gestation. In some embodiments, analyte measurements are performed weekly, biweekly, monthly, per trimester, pre- and post-health event, after delivery, and any combination thereof. The precise collection timeline will depend on the data to be collected and the model to be constructed.

A number of analytes can be used to estimate GA, including (but not limited to) organic acids, organoheterocyclic compounds, lipids and lipid-like molecules, benzenoids, organic oxygen compounds and other minor chemical classes. In some embodiments, clinical data and/or personal data can be additionally used to determine GA. Analytes can be detected and measured by a number of methods, including (but not limited to) mass spectrometry, colorimetric analysis, immunodetection, and the like. It should be noted that static, median, average, and/or dynamic analyte measurements can be used in accordance with various embodiments.

In numerous embodiments, urine samples are collected from individuals that have been diagnosed as being pregnant, as determined by any appropriate method (e.g., ultrasonography). Embodiments are also directed to an individual being one that has not been diagnosed as pregnant.

A collection of individuals, in accordance with many embodiments, is a group of pregnant individuals providing urine samples such that their analytes can be measured and used to construct and train a computational model. The number of individuals in a collection can vary, and in some embodiments, having a greater number of individuals will increase the prediction power of a trained computer model. The precise number and composition of individuals will vary, depending on the model to be constructed and trained.

Based on studies performed, it has been found that several analyte measurements provide robust predictive ability, including (but not limited to) organic acids, organoheterocyclic compounds, lipids and lipid-like molecules, benzenoids, organic oxygen compounds and other minor chemical classes. A number of methods can be used to select analyte measurements to be used as features in the training model. In some embodiments, correlation measurements between analyte measurements and gestational age are used to select features. In various embodiments, a computational model is used to determine which analyte measurements are best predictors. For example, a linear regression model (e.g., LASSO), a random forest regression model, or an elastic net model can be used to determine which analyte measurement features provide the best predictive power as determined by their contribution.

A selection of predictive analyte measurement features are described in the Exemplary Embodiments. For instance, it has been found that the following metabolites provide predictive power and can be utilized within a predictive model: C19H28O8S, C25H34O10, and estriol glucuronide. Based on the foregoing, it should be understood that a number of combinations of analyte features can be used solitarily or combined in any fashion to be used to train a predictive computational model. In some embodiments, a predictive model incorporates measurements of one or more of the following as analyte features: C19H28O8S, C25H34O10, or estriol glucuronide. In some embodiments, a predictive model incorporates measurements of two or more of the following as analyte features: C19H28O8S, C25H34O10, or estriol glucuronide. In some embodiments, a predictive model incorporates measurements of the following as analyte features: C19H28O8S, C25H34O10, and estriol glucuronide.

In some embodiments, a predictive model incorporates measurements of one or more of the following as analyte features: estriol glucuronide, C19H28O8S, C25H34O10, C24H28O7, C24H34O9, C19H26SO7, C14H12N2O4, C24H30O9, or estrone. In some embodiments, a predictive model incorporates measurements of two or more of the following as analyte features: estriol glucuronide, C19H28O8S, C25H34O10, C24H28O7, C24H34O9, C19H26SO7, C14H12N2O4, C24H30O9, or estrone. In some embodiments, a predictive model incorporates measurements of three or more of the following as analyte features: estriol glucuronide, C19H28O8S, C25H34O10, C24H28O7, C24H34O9, C19H26SO7, C14H12N2O4, C24H30O9, or estrone. In some embodiments, a predictive model incorporates measurements of four or more of the following as analyte features: estriol glucuronide, C19H28O8S, C25H34O10, C24H28O7, C24H34O9, C19H26SO7, C14H12N2O4, C24H30O9, or estrone. In some embodiments, a predictive model incorporates measurements of five or more of the following as analyte features: estriol glucuronide, C19H28O8S, C25H34O10, C24H28O7, C24H34O9, C19H26SO7, C14H12N2O4, C24H30O9, or estrone. In some embodiments, a predictive model incorporates measurements of six or more of the following as analyte features: estriol glucuronide, C19H28O8S, C25H34O10, C24H28O7, C24H34O9, C19H26SO7, C14H12N2O4, C24H30O9, or estrone. In some embodiments, a predictive model incorporates measurements of seven or more of the following as analyte features: estriol glucuronide, C19H28O8S, C25H34O10, C24H28O7, C24H34O9, C19H26SO7, C14H12N2O4, C24H30O9, or estrone. In some embodiments, a predictive model incorporates measurements of eight or more of the following as analyte features: estriol glucuronide, C19H28O8S, C25H34O10, C24H28O7, C24H34O9, C19H26SO7, C14H12N2O4, C24H30O9, or estrone. In some embodiments, a predictive model incorporates measurements of the following as analyte features: estriol glucuronide, C19H28O8S, C25H34O10, C24H28O7, C24H34O9, C19H26SO7, C14H12N2O4, C24H30O9, or estrone.

Training labels associating analyte measurement features are used to construct and train (203) a computational model to estimate GA. Various embodiments construct and train a model to determine GA, pregnancy progression and health. A number of models can be used in accordance with various embodiments, including (but not limited to) ridge regression, K-nearest neighbors, LASSO regression, elastic net, least angle regression (LAR), random forest regression, and principal components analysis.

Models and sets of training labels used to train a model can be evaluated for their ability to accurately determine GA. By evaluating models, predictive abilities of analyte measurements can be confirmed. In some embodiments, a portion of the cohort data is withheld to test the model to determine its efficiency and accuracy. A number of accuracy evaluations can be performed, including (but not limited to) area under the receiver operating characteristics (AUROC), R-square error analysis, and root mean square error analysis. In some embodiments, the contribution of each feature to the ability to predict outcome is determined. In some embodiments, top contributing features are utilized to construct the model. Accordingly, an optimized model can be identified.

Process 200 also outputs (205) the parameters of a computational model indicative of GA from a panel of analyte measurements. Computational models can be used to determine GA, inform on disease risk, and on treatment accordingly, as will be described in detail below.

While specific examples of processes for constructing and training a computational model to determine an individual’s GA are described above, one of ordinary skill in the art can appreciate that various steps of the process can be performed in different orders and that certain steps may be optional according to some embodiments. As such, it should be clear that the various steps of the process could be used as appropriate to the requirements of specific applications. Furthermore, any of a variety of processes for constructing and training a computational model appropriate to the requirements of a given application can be utilized in accordance with various embodiments.

### Determination of an Individual’s Pregnancy Progression and Potential Complications using Analyte Measurements

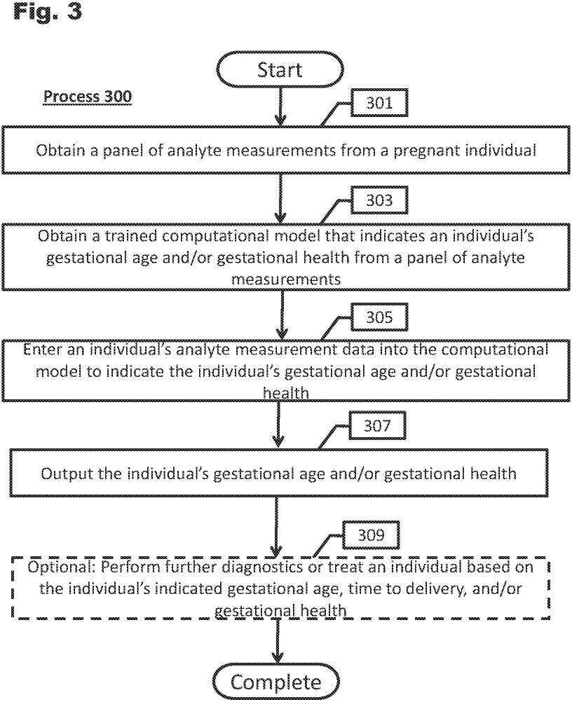

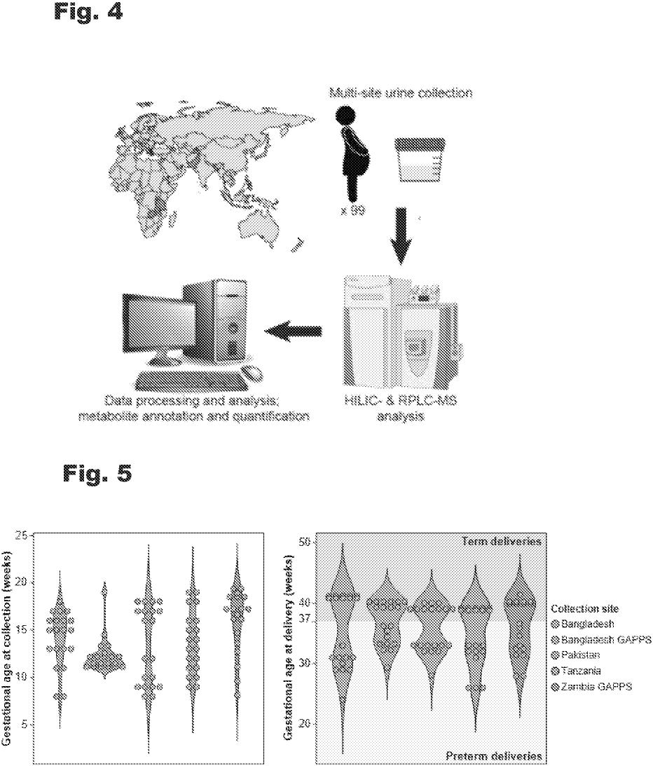

Once a computational model has been constructed and trained, it can be used to compute a determination of an individual’s gestational progress and/or gestational health. As shown in FIG. 3, a method to determine an individual’s gestational progress and/or gestational health using a trained computational model is provided in accordance with various embodiments. Process 300 obtains (301) one or more analyte measurements of analytes collected from a pregnant individual.

In several embodiments, analytes are measured from a urine sample of an individual. In some embodiments, an individual’s sample is collected during fasting. A number of methods are known to collect a sample from an individual and can be used within various embodiments of the invention. In some embodiments, a single urine sample is collected. In some embodiments, the single urine sample is collected before 20 weeks of gestation. In some embodiments, the single urine sample is collected between 8 and 19 weeks of gestation. In some embodiments, analytes are collected and measured at numerous time points, resulting in a dynamic analysis of the analytes. In some of these embodiments, analytes are measured with periodicity (e.g., weekly, monthly, trimester).

A number of analytes can be used to determine GA or gestational health, including (but not limited to) organic acids, organoheterocyclic compounds, lipids and lipid-like molecules, benzenoids, organic oxygen compounds and other minor chemical classes. In some embodiments, clinical data and/or personal data can be additionally used to determine gestational progress and/or preterm birth. Analytes can be detected and measured by a number of methods, including mass spectrometry, colorimetric analysis, immunodetection, and the like. It should be noted that static, median, average, and/or dynamic analyte measurements can be used in accordance with various embodiments. In many embodiments, the precise panel of analytes to be measured depends on the constructed and trained computational model to be used, as the input analyte measurement data that will need to at least partially overlap with the features used to train the model. That is, there should be enough overlap between the feature measurements used to train the model and the individual’s analyte measurements obtained such that gestational age estimation, pregnancy progression and/or gestational health can be determined.

In some embodiments, an individual has been diagnosed as being pregnant, as determined by any appropriate method (e.g., ultrasonography or urine or blood test). Embodiments are also directed to an individual being one that has not been diagnosed as pregnant, especially in situations in which the individual is unaware of her pregnancy.

Process 300 also obtains (303) a trained computational model that indicates an individual’s GA and/or gestational health from a panel of analyte measurements. Any computational model that can compute an indicator of an individual’s GA and/or gestational health from a panel of analyte measurements can be used. In some embodiments, the computational model is constructed and trained as described in FIG. 2. The computational model, in accordance with various embodiments, has been optimized to accurately and efficiently estimate GA.

A number of models can be used in accordance with various embodiments, including (but not limited to) ridge regression, K-nearest neighbors, LASSO regression, elastic net, least angle regression (LAR), random forest regression, and principal components analysis.

Process 300 also enters (305) an individual’s analyte measurement data into a computational model to indicate the individual’s gestational age. In some embodiments, the analyte measurement data is used to compute an individual’s gestational age in lieu of performing a traditional gestational analysis (e.g., ultrasonography). Various embodiments utilize the analyte measurement data and computational model in combination with a clinical diagnostic method.

Based on studies performed, it has been found that several analyte measurements provide robust predictive ability, including (but not limited to) organic acids, organoheterocyclic compounds, lipids and lipid-like molecules, benzenoids, organic oxygen compounds and other minor chemical classes. A number of methods can be used to select analyte measurements to be used as features in the training model. In some embodiments, correlation measurements between analyte measurements and GA are used to select features. In various embodiments, a computational model is used to determine which analyte measurements are best predictors. For example, a linear regression model (e.g., LASSO), random forest regression model, or elastic net model can be used to determine which analyte measurement features provide the best predictive power as determined by their contribution.

A selection of predictive analyte measurement features are described in the Exemplary Embodiments. For instance, it has been found that the following metabolites provide predictive power and can be utilized within a predictive model: C19H28O8S, C25H34O10, and estriol glucuronide. In some embodiments, a predictive model incorporates measurements of one or more of the following as analyte features: C19H28O8S, C25H34O10, or estriol glucuronide. In some embodiments, a predictive model incorporates measurements of two or more of the following as analyte features: C19H28O8S, C25H34O10, or estriol glucuronide. In some embodiments, a predictive model incorporates measurements of the following as analyte features: C19H28O8S, C25H34O10, and estriol glucuronide.

In some embodiments, a predictive model incorporates measurements of one or more of the following as analyte features: estriol glucuronide, C19H28O8S, C25H34O10, C24H28O7, C24H34O9, C19H26SO7, C14H12N2O4, C24H30O9, or estrone. In some embodiments, a predictive model incorporates measurements of two or more of the following as analyte features: estriol glucuronide, C19H28O8S, C25H34O10, C24H28O7, C24H34O9, C19H26SO7, C14H12N2O4, C24H30O9, or estrone. In some embodiments, a predictive model incorporates measurements of three or more of the following as analyte features: estriol glucuronide, C19H28O8S, C25H34O10, C24H28O7, C24H34O9, C19H26SO7, C14H12N2O4, C24H30O9, or estrone. In some embodiments, a predictive model incorporates measurements of four or more of the following as analyte features: estriol glucuronide, C19H28O8S, C25H34O10, C24H28O7, C24H34O9, C19H26SO7, C14H12N2O4, C24H30O9, or estrone. In some embodiments, a predictive model incorporates measurements of five or more of the following as analyte features: estriol glucuronide, C19H28O8S, C25H34O10, C24H28O7, C24H34O9, C19H26SO7, C14H12N2O4, C24H30O9, or estrone. In some embodiments, a predictive model incorporates measurements of six or more of the following as analyte features: estriol glucuronide, C19H28O8S, C25H34O10, C24H28O7, C24H34O9, C19H26SO7, C14H12N2O4, C24H30O9, or estrone. In some embodiments, a predictive model incorporates measurements of seven or more of the following as analyte features: estriol glucuronide, C19H28O8S, C25H34O10, C24H28O7, C24H34O9, C19H26SO7, C14H12N2O4, C24H30O9, or estrone. In some embodiments, a predictive model incorporates measurements of eight or more of the following as analyte features: estriol glucuronide, C19H28O8S, C25H34O10, C24H28O7, C24H34O9, C19H26SO7, C14H12N2O4, C24H30O9, or estrone. In some embodiments, a predictive model incorporates measurements of the following as analyte features: estriol glucuronide, C19H28O8S, C25H34O10, C24H28O7, C24H34O9, C19H26SO7, C14H12N2O4, C24H30O9, or estrone.

Process 300 also outputs (307) a report containing an individual’s gestational age, weeks to delivery, and/or gestational health result and/or diagnosis. Furthermore, based on an individual’s indicated gestational progress and/or gestational health, the individual is optionally further examined and/or treated (309) to ameliorate a symptom related to the result and/or diagnosis. In several embodiments, an individual is provided with a personalized treatment plan. Further discussion of treatments that can be utilized in accordance with this embodiment are described in detail below, which may include various medications, dietary supplements, and surgical procedures.

While specific examples of processes for determining an individual’s GA and/or gestational health are described above, one of ordinary skill in the art can appreciate that various steps of the process can be performed in different orders and that certain steps may be optional according to some embodiments. As such, it should be clear that the various steps of the process could be used as appropriate to the requirements of specific applications. Furthermore, any of a variety of processes for computing an individual’s GA and/or gestational health is appropriate to the requirements of a given application can be utilized in accordance with various embodiments.

### Feature Selection

As explained in the previous sections, analyte measurements are used as features to construct a computational model that is then used to indicate an individual’s GA and/or gestational health. Analyte measurement features used to train the model can be selected by a number of ways. In some embodiments, analyte measurement features are determined by which measurements provide strong correlation with gestational age. In various embodiments, analyte measurement features are determined using a computational model, such as Bayesian network, which can determine which analyte measurements influence or are influenced by an individual’s GA. Embodiments also consider practical factors, such as (for example) the ease and/or cost of obtaining the analyte measurement, patient comfort when obtaining the biological sample and/or analyte measurement, and current clinical protocols are also considered when selecting features.

Correlation analysis utilizes statistical methods to determine the strength of relationships between two measurements. Accordingly, a strength of relationship between an analyte measurement and gestational progress and/or gestational health can be determined. Many statistical methods are known to determine correlation strength (e.g., correlation coefficient), including linear association (Pearson correlation coefficient), Kendall rank correlation coefficient, and Spearman rank correlation coefficient. Analyte measurements that correlate strongly with gestational age can then be used as features to construct a computational model to determine an individual’s gestational progress and/or gestational health.

In a number of embodiments, analyte measurement features are identified by a computational model, including (but not limited to) a Bayesian network model, LASSO, random forest and elastic net. In some embodiments, the contribution of a feature to the predictive ability of the model is determined and features are selected based on their contribution. In some embodiments, the top contributing features are utilized. The precise number of contributing features will depend on the results of the model and each feature’s contribution. Various embodiments utilize an appropriate computational model that results in a number of features that is manageable. For instance, constructing predictive models from hundreds to thousands of analyte measurement features may have overfitting issues. Likewise, too few features can result in less prediction power.

### Biomarkers as Indicators of Gestation Age and Health

In several embodiments, biomarkers are detected and measured, and based on the ability to be detected and/or level of the biomarker, gestational age and/or gestational health can be determined directly or via a computational model. Biomarkers that can be used in the practice of the invention include (but are not limited to) metabolites, protein constituents, genomic DNA, transcript expression, and lipids. As discussed in the Exemplary embodiments, a number of biomarkers have been found to be useful to determine gestational age, including (but not limited to) C19H28O8S, C25H34O10, and estriol glucuronide.

### Detecting and Measuring Levels of Biomarkers

Analyte biomarkers in a biological sample (e.g., urine sample) can be determined by a number of suitable methods. Suitable methods include chromatography (e.g., high-performance liquid chromatography (HPLC), gas chromatography (GC), liquid chromatography (LC)), mass spectrometry (e.g., MS, MS/MS), NMR, enzymatic or biochemical reactions, immunoassay, and combinations thereof. For example, mass spectrometry can be combined with chromatographic methods, such as liquid chromatography (LC), gas chromatography (GC), or electrophoresis to separate the metabolite being measured from other components in the biological sample. See, e.g., Hyotylainen (2012) Expert Rev. Mol. Diagn. 12(5):527-538; Beckonert et al. (2007) Nat. Protoc. 2(11):2692-2703; O’Connell (2012) Bioanalysis 4(4):431-451; and Eckhart et al. (2012) Clin. Transl. Sci. 5(3):285-288; the disclosures of which are herein incorporated by reference. Alternatively, analytes can be measured with biochemical or enzymatic assays. For example, glucose can be measured with a hexokinase-glucose-6-phosphate dehydrogenase coupled enzyme assay. In another example, biomarkers can be separated by chromatography and relative levels of a biomarker can be determined from analysis of a chromatogram by integration of the peak area for the eluted biomarker.

Immunoassays based on the use of antibodies that specifically recognize a biomarker may be used for measurement of biomarker levels. Such assays include (but are not limited to) enzyme-linked immunosorbent assay (ELISA), radioimmunoassays (RIA), “sandwich” immunoassays, fluorescent immunoassays, enzyme multiplied immunoassay technique (EMIT), capillary electrophoresis immunoassays (CEIA), immunoprecipitation assays, western blotting, immunohistochemistry (IHC), flow cytometry, and cytometry by time of flight (CyTOF).

Antibodies that specifically bind to a biomarker can be prepared using any suitable methods known in the art. See, e.g., Coligan, Current Protocols in Immunology (1991); Harlow & Lane, Antibodies: A Laboratory Manual (1988); Goding, Monoclonal Antibodies: Principles and Practice (2d ed. 1986); and Kohler & Milstein, Nature 256:495-497 (1975). A biomarker antigen can be used to immunize a mammal, such as a mouse, rat, rabbit, guinea pig, monkey, or human, to produce polyclonal antibodies. If desired, a biomarker antigen can be conjugated to a carrier protein, such as bovine serum albumin, thyroglobulin, and keyhole limpet hemocyanin. Depending on the host species, various adjuvants can be used to increase the immunological response. Such adjuvants include, but are not limited to, Freund’s adjuvant, mineral gels (e.g., aluminum hydroxide), and surface-active substances (e.g. lysolecithin, pluronic polyols, polyanions, peptides, oil emulsions, keyhole limpet hemocyanin, and dinitrophenol). Among adjuvants used in humans, BCG (bacilli Calmette-Guerin) and Corynebacterium parvum are especially useful.

Monoclonal antibodies which specifically bind to a biomarker antigen can be prepared using any technique which provides for the production of antibody molecules by continuous cell lines in culture. These techniques include, but are not limited to, the hybridoma technique, the human B cell hybridoma technique, and the EBV hybridoma technique (Kohler et al., Nature 256, 495-97, 1985; Kozbor et al., J. Immunol. Methods 81, 31 42, 1985; Cote et al., Proc. Natl. Acad. Sci. 80, 2026-30, 1983; Cole et al., Mol. Cell Biol. 62, 109-20, 1984).

In addition, techniques developed for the production of “chimeric antibodies,” the splicing of mouse antibody genes to human antibody genes to obtain a molecule with appropriate antigen specificity and biological activity, can be used (Morrison et al., Proc. Natl. Acad. Sci. 81, 6851-55, 1984; Neuberger et al., Nature 312, 604-08, 1984; Takeda et al., Nature 314, 452-54, 1985). Monoclonal and other antibodies also can be “humanized” to prevent a patient from mounting an immune response against the antibody when it is used therapeutically. Such antibodies may be sufficiently similar in sequence to human antibodies to be used directly in therapy or may require alteration of a few key residues. Sequence differences between rodent antibodies and human sequences can be minimized by replacing residues which differ from those in the human sequences by site directed mutagenesis of individual residues or by grating of entire complementarity determining regions.

Alternatively, humanized antibodies can be produced using recombinant methods, as described below. Antibodies which specifically bind to a particular antigen can contain antigen binding sites which are either partially or fully humanized, as disclosed in U.S. Pat. No. 5,565,332. Human monoclonal antibodies can be prepared in vitro as described in Simmons et al., PLoS Medicine 4(5), 928-36, 2007.

Alternatively, techniques described for the production of single chain antibodies can be adapted using methods known in the art to produce single chain antibodies which specifically bind to a particular antigen. Antibodies with related specificity, but of distinct idiotypic composition, can be generated by chain shuffling from random combinatorial immunoglobin libraries (Burton, Proc. Natl. Acad. Sci. 88, 11120-23, 1991).

Single-chain antibodies also can be constructed using a DNA amplification method, such as PCR, using hybridoma cDNA as a template (Thirion et al., Eur. J. Cancer Prev. 5, 507-11, 1996). Single-chain antibodies can be mono- or bispecific, and can be bivalent or tetravalent. Construction of tetravalent, bispecific single-chain antibodies is taught, for example, in Coloma & Morrison, Nat. Biotechnol. 15, 159-63, 1997. Construction of bivalent, bispecific single-chain antibodies is taught in Mallender & Voss, J. Biol. Chem. 269, 199-206, 1994.

A nucleotide sequence encoding a single-chain antibody can be constructed using manual or automated nucleotide synthesis, cloned into an expression construct using standard recombinant DNA methods, and introduced into a cell to express the coding sequence, as described below. Alternatively, single-chain antibodies can be produced directly using, for example, filamentous phage technology (Verhaar et al., Int. J Cancer 61, 497-501, 1995; Nicholls et al., J. Immunol. Meth. 165, 81-91, 1993).

Antibodies which specifically bind to a biomarker antigen also can be produced by inducing in vivo production in the lymphocyte population or by screening immunoglobulin libraries or panels of highly specific binding reagents as disclosed in the literature (Orlandi et al., Proc. Natl. Acad. Sci. 86, 3833 3837, 1989; Winter et al., Nature 349, 293 299, 1991).

Chimeric antibodies can be constructed as disclosed in WO 93/03151. Binding proteins which are derived from immunoglobulins and which are multivalent and multispecific, such as the “diabodies” described in WO 94/13804, also can be prepared.

Antibodies can be purified by methods well known in the art. For example, antibodies can be affinity purified by passage over a column to which the relevant antigen is bound. The bound antibodies can then be eluted from the column using a buffer with a high salt concentration.

Antibodies may be used in diagnostic assays to detect the presence or for quantification of the biomarkers in a biological sample. Such a diagnostic assay may comprise at least two steps; (i) contacting a biological sample with the antibody, wherein the sample is blood or plasma, a microchip (e.g., See Kraly et al. (2009) Anal Chim Acta 653(1):23-35), or a chromatography column with bound biomarkers, etc.; and (ii) quantifying the antibody bound to the substrate. The method may additionally involve a preliminary step of attaching the antibody, either covalently, electrostatically, or reversibly, to a solid support, before subjecting the bound antibody to the sample, as defined above and elsewhere herein.

Various diagnostic assay techniques are known in the art, such as competitive binding assays, direct or indirect sandwich assays and immunoprecipitation assays conducted in either heterogeneous or homogenous phases (Zola, Monoclonal Antibodies: A Manual of Techniques, CRC Press, Inc., (1987), pp 147-158). The antibodies used in the diagnostic assays can be labeled with a detectable moiety. The detectable moiety should be capable of producing, either directly or indirectly, a detectable signal. For example, the detectable moiety may be a radioisotope, such as 2H, 14C, 32P, or 125I, a florescent or chemiluminescent compound, such as fluorescein isothiocyanate, rhodamine, or luciferin, or an enzyme, such as alkaline phosphatase, beta-galactosidase, green fluorescent protein, or horseradish peroxidase. Any method known in the art for conjugating the antibody to the detectable moiety may be employed, including those methods described by Hunter et al., Nature, 144:945 (1962); David et al., Biochem. 13:1014 (1974); Pain et al., J. Immunol. Methods 40:219 (1981); and Nygren, J. Histochem. and Cytochem. 30:407 (1982).

Immunoassays can be used to determine the presence or absence of a biomarker in a sample as well as the quantity of a biomarker in a sample. First, a test amount of a biomarker in a sample can be detected using the immunoassay methods described above. If a biomarker is present in the sample, it will form an antibody-biomarker complex with an antibody that specifically binds the biomarker under suitable incubation conditions, as described above. The amount of an antibody-biomarker complex can be determined by comparing to a standard. A standard can be, e.g., a known compound or another protein known to be present in a sample. As noted above, the test amount of a biomarker need not be measured in absolute units, as long as the unit of measurement can be compared to a control.

In various embodiments, biomarkers in a sample can be separated by high-resolution electrophoresis, e.g., one or two-dimensional gel electrophoresis. A fraction containing a biomarker can be isolated and further analyzed by gas phase ion spectrometry. Preferably, two-dimensional gel electrophoresis is used to generate a two-dimensional array of spots for the biomarkers. See, e.g., Jungblut and Thiede, Mass Spectr. Rev. 16:145-162 (1997).

Two-dimensional gel electrophoresis can be performed using methods known in the art. See, e.g., Deutscher ed., Methods In Enzymology vol. 182. Typically, biomarkers in a sample are separated by, e.g., isoelectric focusing, during which biomarkers in a sample are separated in a pH gradient until they reach a spot where their net charge is zero (i.e., isoelectric point). This first separation step results in one-dimensional array of biomarkers. The biomarkers in the one-dimensional array are further separated using a technique generally distinct from that used in the first separation step. For example, in the second dimension, biomarkers separated by isoelectric focusing are further resolved using a polyacrylamide gel by electrophoresis in the presence of sodium dodecyl sulfate (SDS-PAGE). SDS-PAGE allows further separation based on molecular mass. Typically, two-dimensional gel electrophoresis can separate chemically different biomarkers with molecular masses in the range from 1000-200,000 Da, even within complex mixtures.

Biomarkers in the two-dimensional array can be detected using any suitable methods known in the art. For example, biomarkers in a gel can be labeled or stained (e.g., Coomassie Blue or silver staining). If gel electrophoresis generates spots that correspond to the molecular weight of one or more biomarkers of the invention, the spot can be further analyzed by densitometric analysis or gas phase ion spectrometry. For example, spots can be excised from the gel and analyzed by gas phase ion spectrometry. Alternatively, the gel containing biomarkers can be transferred to an inert membrane by applying an electric field. Then a spot on the membrane that approximately corresponds to the molecular weight of a biomarker can be analyzed by gas phase ion spectrometry. In gas phase ion spectrometry, the spots can be analyzed using any suitable techniques, such as MALDI or SELDI.

In a number of embodiments, high performance liquid chromatography (HPLC) can be used to separate a mixture of biomarkers in a sample based on their different physical properties, such as polarity, charge and size. HPLC instruments typically consist of a reservoir, the mobile phase, a pump, an injector, a separation column, and a detector. Biomarkers in a sample are separated by injecting an aliquot of the sample onto the column. Different biomarkers in the mixture pass through the column at different rates due to differences in their partitioning behavior between the mobile liquid phase and the stationary phase. A fraction that corresponds to the molecular weight and/or physical properties of one or more biomarkers can be collected. The fraction can then be analyzed by gas phase ion spectrometry to detect biomarkers.

After preparation, biomarkers in a sample are typically captured on a substrate for detection. Traditional substrates include antibody-coated 96-well plates or nitrocellulose membranes that are subsequently probed for the presence of biomarkers. Alternatively, metabolite-binding molecules attached to microspheres, microparticles, microbeads, beads, or other particles can be used for capture and detection of biomarkers. The metabolite-binding molecules may be antibodies, peptides, peptoids, aptamers, small molecule ligands or other metabolite-binding capture agents attached to the surface of particles. Each metabolite-binding molecule may comprise a “unique detectable label,” which is uniquely coded such that it may be distinguished from other detectable labels attached to other metabolite-binding molecules to allow detection of biomarkers in multiplex assays. Examples include, but are not limited to, color-coded microspheres with known fluorescent light intensities (see e.g., microspheres with xMAP technology produced by Luminex (Austin, TX); microspheres containing quantum dot nanocrystals, for example, having different ratios and combinations of quantum dot colors (e.g., Qdot nanocrystals produced by Life Technologies (Carlsbad, CA); glass coated metal nanoparticles (see e.g., SERS nanotags produced by Nanoplex Technologies, Inc. (Mountain View, CA); barcode materials (see e.g., sub-micron sized striped metallic rods such as Nanobarcodes produced by Nanoplex Technologies, Inc.), encoded microparticles with colored bar codes (see e.g., CellCard produced by Vitra Bioscience, vitrabio.com), glass microparticles with digital holographic code images (see e.g., CyVera microbeads produced by Illumina (San Diego, CA); chemiluminescent dyes, combinations of dye compounds; and beads of detectably different sizes. See, e.g., U.S. patent No. 5,981,180, U.S. Pat. No. 7,445,844, U.S. Pat. No. 6,524,793, Rusling et al. (2010) Analyst 135(10): 2496-2511; Kingsmore (2006) Nat. Rev. Drug Discov. 5(4): 310-320, Proceedings Vol. 5705 Nanobiophotonics and Biomedical Applications II, Alexander N. Cartwright; Marek Osinski, Editors, pp.114-122; Nanobiotechnology Protocols Methods in Molecular Biology, 2005, Volume 303; herein incorporated by reference in their entireties).

Mass spectrometry, and particularly SELDI mass spectrometry, is useful for detection of biomarkers. Laser desorption time-of-flight mass spectrometer can be used in embodiments of the invention. In laser desorption mass spectrometry, a substrate or a probe comprising biomarkers is introduced into an inlet system. The biomarkers are desorbed and ionized into the gas phase by laser from the ionization source. The ions generated are collected by an ion optic assembly, and then in a time-of-flight mass analyzer, ions are accelerated through a short high voltage field and let drift into a high vacuum chamber. At the far end of the high vacuum chamber, the accelerated ions strike a sensitive detector surface at a different time. Since the time-of-flight is a function of the mass of the ions, the elapsed time between ion formation and ion detector impact can be used to identify the presence or absence of markers of specific mass to charge ratio.

Matrix-assisted laser desorption/ionization mass spectrometry (MALDI-MS) can also be used for detecting biomarkers. MALDI-MS is a method of mass spectrometry that involves the use of an energy absorbing molecule, frequently called a matrix, for desorbing proteins intact from a probe surface. MALDI is described, for example, in U.S. Pat. No. 5,118,937 (Hillenkamp et al.) and U.S. Pat. No. 5,045,694 (Beavis and Chait). In MALDI-MS, the sample is typically mixed with a matrix material and placed on the surface of an inert probe. Exemplary energy absorbing molecules include cinnamic acid derivatives, sinapinic acid (“SPA”), cyano hydroxy cinnamic acid (“CHCA”) and dihydroxybenzoic acid. Other suitable energy absorbing molecules are known to those skilled in this art. The matrix dries, forming crystals that encapsulate the analyte molecules. Then the analyte molecules are detected by laser desorption/ionization mass spectrometry.

Biomarkers on the substrate surface can be desorbed and ionized using gas phase ion spectrometry. Any suitable gas phase ion spectrometer can be used as long as it allows biomarkers on the substrate to be resolved. Preferably, gas phase ion spectrometers allow quantitation of biomarkers. In one embodiment, a gas phase ion spectrometer is a mass spectrometer. In a typical mass spectrometer, a substrate or a probe comprising biomarkers on its surface is introduced into an inlet system of the mass spectrometer. The biomarkers are then desorbed by a desorption source such as a laser, fast atom bombardment, high energy plasma, electrospray ionization, thermospray ionization, liquid secondary ion MS, field desorption, etc. The generated desorbed, volatilized species consist of preformed ions or neutrals which are ionized as a direct consequence of the desorption event. Generated ions are collected by an ion optic assembly, and then a mass analyzer disperses and analyzes the passing ions. The ions exiting the mass analyzer are detected by a detector. The detector then translates information of the detected ions into mass-to-charge ratios. Detection of the presence of biomarkers or other substances will typically involve detection of signal intensity. This, in turn, can reflect the quantity and character of biomarkers bound to the substrate. Any of the components of a mass spectrometer (e.g., a desorption source, a mass analyzer, a detector, etc.) can be combined with other suitable components described herein or others known in the art in embodiments of the invention.

The methods for detecting biomarkers in a sample have many applications. For example, the biomarkers are useful in monitoring women during pregnancy, for example to determine gestational age, predict time until delivery, or assess risk of spontaneous abortion.

### Kits

In several embodiments, kits are utilized for monitoring individuals during pregnancy, wherein the kits can be used to detect analyte biomarkers as described herein. For example, the kits can be used to detect any one or more of the analyte biomarkers described herein, which can be used to determine gestational age and/or gestational health. The kit may include one or more agents for detection of one or more metabolite biomarkers, a container for holding a biological sample (e.g., urine) obtained from a subject; and printed instructions for reacting agents with the biological sample to detect the presence or amount of one or more biomarkers in the sample. The agents may be packaged in separate containers. The kit may further comprise one or more control reference samples and reagents for performing a biochemical assay, enzymatic assay, immunoassay, or chromatography. In various embodiments, a kit may include an antibody that specifically binds to a biomarker. In some embodiments, a kit may contain reagents for performing liquid chromatography (e.g., resin, solvent, and/or column).

A kit can include one or more containers for compositions contained in the kit. Compositions can be in liquid form or can be lyophilized. Suitable containers for the compositions include, for example, bottles, vials, syringes, and test tubes. Containers can be formed from a variety of materials, including glass or plastic. The kit can also comprise a package insert containing written instructions for methods of monitoring individual during pregnancy, e.g., to determine gestational age and/or gestational health.

### Applications and Treatments Related to Gestational Progress and Health

Various embodiments are directed to performing further diagnostics and or treatments based on a determination of gestational age and/or gestational health. As described herein, a pregnant individual’s pregnancy progression and/or likelihood of developing a condition is determined by various methods (e.g., computational methods, biomarkers). Based on one’s GA and/or likelihood of developing a condition, an individual can be subjected to further diagnostic testing and/or treated with various medications, dietary supplements, and surgical procedures.

### Clinical Diagnostics, Medications and Supplements

Several embodiments are directed to the use of medications and/or dietary supplements to treat an individual based on their gestational age and/or gestational health determination. In some embodiments, medications and/or dietary supplements are administered in a therapeutically effective amount as part of a course of treatment. As used in this context, to “treat” means to ameliorate at least one symptom of the disorder to be treated or to provide a beneficial physiological effect. For example, one such amelioration of a symptom could be improvement in gestational health. Assessment of gestational progress and/or gestational health can be performed in many ways, including (but not limited to) the use of analyte measurements and sonography.

A therapeutically effective amount can be an amount sufficient to prevent reduce, ameliorate or eliminate the symptoms of diseases or pathological conditions susceptible to such treatment, such as, for example, preterm birth or other gestational disorders. In some embodiments, a therapeutically effective amount is an amount sufficient to improve gestational health or reduce the risk of premature delivery.

Various embodiments are directed towards getting an indication of gestational progress and performing an intervention and/or treatment thereupon. In some embodiments, when a pregnant individual is experiencing various symptoms at various points of gestational age or timeline to pregnancy (as determined by methods described herein), an intervention and/or treatment is performed. In some embodiments, treatments are performed when an individual exhibits symptoms that occur early and/or late according a determined gestational age or timeline to delivery. For example, a pregnant individual experiencing regular contractions prior to 37 weeks is considered to be in premature (preterm) labor, and a number of interventions and/or treatments can be performed. Likewise, gestation periods of longer than 42 weeks is considered to be a postterm pregnancy, additional monitoring, induction of labor, and/or caesarian delivery is performed to avoid complications.

In a number of embodiments, when a pregnant individual is experiencing regular contractions, a gestational age can be determined, which would indicate whether the individual is experiencing preterm labor. In some embodiments, a gestational age is determined prior to any experienced contractions (e.g., as determined during the course of pregnancy) and based on the determined gestational age, an indication of preterm labor is determined. In accordance with various embodiments, it may be desirable to confirm that an individual is in preterm labor, and thus confirmation of labor can be performed by a number of means, including (but not limited to) cervical exam, sonography, testing for amniotic fluid, testing for fetal fibronectin, or any combination thereof. Treatments for preterm labor include (but not limited to) intravenous fluids, antibiotics (to treat infection), tocolytic medications (to slow or stop contractions), antenatal corticosteroids (to help mature fetus), cervical cerclage (to close up cervix), delivery of the baby, or any appropriate combination thereof. Tocolytic medications include (but not limited to) indomethacin, magnesium sulfate, orciprenaline, ritodrine, terbutaline, salbutamol, nifedipine, fenoterol, nylidrin, isoxsuprine, hexoprenaline, and atosiban. Antenatal corticosteroids include (but not limited to) dexamethasone and betamethasone. For more on treatment and care of preterm labor, see J. N. Robinson and E. R. Norwitz. Ed.: V. A. Barss. UpToDate, retrieved September 2019 (https://www.uptodate.com/contents/preterm-birth-risk-factors-interventions-for-risk-reduction-and-maternal-prognosis); C. J. Lockwood. Ed.: V. A. Barss. UpToDate, retrieved September 2019 (https://www.uptodate.com/contents/preterm-labor-clinical-findings-diagnostic-evaluation-and-initial-treatment); and H. N. Simhan and S. Caritis. Ed.: V. A. Barss. UpToDate, retrieved September 2019 (https://www.uptodate.com/contents/inhibition-of-acute-preterm-labor); the disclosure of which are each incorporated herein by reference).

In several embodiments, a pregnancy may go beyond a gestational age of 42 weeks, as determined by various methods described herein. As gestational age exceeds 42 weeks, the placenta may age, begin deteriorating, or fail. Accordingly, a number of embodiments are directed towards determining a gestational age and determine whether the individual is in a postterm pregnancy. In some embodiments, when a postterm pregnancy is indicated, additional monitoring can be performed, including (but not limited to) fetal movement recording (to monitor regular movements of fetus), doppler fetal monitor (to measure fetal heart rate), nonstress test (to monitor fetal heartbeat) and Doppler flow study (to monitor blood flow in and out of placenta). In some embodiments, when a postterm pregnancy is indicated, labor is induced and/or Caesarian delivery is performed.

In many embodiments, the gestational age and time to delivery are determined and used concurrently to determine whether an individual will experience preterm labor or a postterm pregnancy. In some embodiments, a time to delivery equal to or less than a gestational age of 37 weeks is determined, indicating that preterm labor is likely and thus interventions and treatments for preterm labor are performed. Likewise, in some embodiments, a time to delivery equal to or more than a gestational age of 42 weeks is determined, indicating that a postterm pregnancy is likely and thus monitoring, induced labor, or Casesarian delivery are performed.

In a similar manner, interventions and/or treatments can be performed at various other time points, as would be understood in the art. Accordingly, various methods described herein can determine gestational progress and based on symptoms, can perform an intervention and/or a treatment. Critical time points include gestational ages of 20 weeks for determination of successful pregnancy and mitigating miscarriage, 24 weeks for determination age of viability, 28 weeks for determination of extreme preterm labor, 32 weeks for very preterm labor, 37 weeks for preterm labor, and 42 weeks for postterm pregnancy. At each time point, various interventions include prenatal checkups and monitoring, including measuring blood pressure, checking for urinary tract infection, checking for signs of preeclampsia, checking for signs of gestational hypertension, checking for signs of gestational diabetes, checking for signs of preterm labor, checking for signs of preterm rupture of membranes, measure heartbeat of fetus, measure fundal height, look for swelling in hands or feet, sampling for chorionic villus, check for risk of genetic disorders (e.g., Down syndrome and spina bifida), perform amniocentesis test, sonography, determine baby gender, and performing blood tests (e.g., glucose screening, anemia, status of Rh-positive or Rh-negative).

### EXEMPLARY EMBODIMENTS

Bioinformatic and biological data support the methods and systems of assessing gestational progress and applications thereof. In the attached manuscript, exemplary methods and exemplary applications related to gestation that incorporate analyte panels, correlations, and computational models are provided.

### Prediction of Gestational Age Using Urinary Metabolites

Human pregnancy involves a myriad of interconnected biological processes that are precisely regulated to ensure proper fetal development and growth. A reliable estimation of gestational age (GA) is critical to provide optimal care for the expectant mother and inform clinical decisions, especially in pregnancies with pathological conditions such as intrauterine growth restriction (IUGR) and preterm birth (PTB). In current clinical practice, GA is best estimated by fetal ultrasound performed before 13 weeks of gestation. However, early ultrasound is often not feasible in resource-limited settings due to later presentation to care or lack of equipment and trained sonographers. Alternatively, GA can be estimated using the reported first day of the last menstrual period (LMP) or various maternal and fetal biometrics, but these methods have been shown to be imprecise or even biased, stressing the need to develop alternative ways to estimate GA. Misclassifications of GA result in inaccurate estimations of prematurity, a major cause of neonatal mortality in South Asia and sub-Saharian Africa. The study of risk factors of prematurity and its impact on long-term outcomes is also impeded by the absence of reliable measures of GA.

Recent omic studies performed in blood have successfully characterized the timing of biological processes during healthy pregnancy and revealed precisely tuned chronological changes at the level of cell-free maternal RNA, immune cells, plasma proteins, and metabolites (see W. Pan, et al., Clin Chem. 2017; 63:1695-704; N. Aghaeepour, et al., Sci Immunol. 2017; 2:eaan2946; N. Aghaeepour, et al., Am J Obstet Gynecol. 2018; 218:347 e1-e14; M. Ghaemi, et al., Bioinformatics. 2019 Jan 1;35(1):95-103; and L. Liang, et al., Cell. 2020; 181:1680-92; the disclosures of which are each incorporated herein by reference). These observations have unveiled a potential utility of blood molecular constituents towards more accurate estimations of GA. While most omic layers demonstrated predictive value, metabolomics - the comprehensive study of metabolites - was among the most performant with steroid hormones and their derivatives being the best predictors. Despite the many advantages of urine as a clinical sample (e.g. non-invasive collection, sterile, and largely-free from interfering proteins and complex lipids), the feasibility of predicting GA using urinary metabolite levels remains unexplored.

In this example, metabolites were profiled using an untargeted liquid chromatography coupled with mass spectrometry (LC-MS) platform in urine samples collected in early pregnancy (8-19 weeks) from women across multiple international study sites. Using random forest (RF) machine learning, it was demonstrated that a small subset of urinary metabolites can predict GA with high precision and accuracy. Metabolites selected in the model informed on individual molecules and biological processes that associated with pregnancy progression. It was found that GA was not predicted as accurately among women who went on to deliver preterm, which was explained in part by a larger inter-individual variability of predictive metabolites in this population.

### Methods

### Study Design and Gestational Age Assessment

Ninety-nine pregnant women were selected for the study and included 20 participants from each site with half delivering preterm (<37 weeks’ GA) and half delivering at term (≥37 weeks’ GA). Only 9 samples were provided from term pregnancies at the Zambia site. Women with multiple births, pre-eclampsia, congenital malformations, stillbirth, or induction of labor for any cause were excluded. Outcomes were assessed through either study procedures on the labor ward or, among those delivering elsewhere, through participant interview via direct phone calls, household visits, and/or medical record review at a postnatal visit.

The study was comprised of a single urine sample for each participant (n = 99) that was collected at a prenatal visit after ultrasound confirmed at < 20 weeks of gestation. Ultrasound imaging was performed by trained sonologists and GA was estimated following guidelines from the American College of Obstetricians and Gynecologists (Bangladesh GAPPS) and using INTERGROWTH-21^{st} equations (Zambia) or Hadlock’s formulas (all AMANHI sites: Bangladesh, Pakistan, Tanzania) (for more on guidelines, see Committee Opinion No 700: Methods for Estimating the Due Date. Obstet Gynecol. 2017; 129:e150-e154; K. Contrepois, L. Jiang, and M. Snyder, Mol Cell Proteomics. 2015; 14:1684-95; E. A. Kuijper, et al., Hum Reprod Update. 2019 Sep 11;25(5):592-632; and D. S. Reddy Trends Pharmacol Sci. 2003; 24:103-6; the disclosures of which are each incorporated herein by referenc). GA was reported in weeks. All study sites employed a uniform method for urine collection and handling. Urine samples were aliquoted and frozen at -80° C. within 2 hours. Deidentified urine aliquots were shipped on dry ice from each biorepository to Stanford University as a single batch and under continuous temperature monitoring. Urine samples from 20 healthy pregnancies collected between 8 and 19 weeks of gestation at the Lucile Packard Children’s Hospital at Stanford University, served as the validation cohort.

### Untargeted Identification of Metabolomics of Urine by Liquid Chromatography (LC)- Mass Spectrometry (MS)

LC-MS-grade solvents and mobile phase modifiers were obtained from Fisher Scientific (water, acetonitrile, methanol) and Sigma-Aldrich (acetic acid, ammonium acetate). Urine samples were analyzed using a broad-spectrum metabolomics platform consisting of hydrophilic interaction chromatography (HILIC) and reverse phase liquid chromatography (RPLC)-MS.

Frozen urine samples were thawed on ice and centrifuged at 17,000 g for 10 min at 4° C. Supernatants (25 µl) were then diluted 1:4 with 75% acetonitrile and 100% water for HILIC- and RPLC-MS experiments, respectively. Each sample was spiked-in with 15 analytical-grade internal standards (IS). Samples for HILIC-MS experiments were further centrifuged at 21,000 g for 10 min at 4° C. to precipitate proteins.

Metabolic extracts were analyzed using HILIC and RPLC separations in both positive and negative ionization modes. Data were acquired on a Thermo Q Exactive HF mass spectrometer equipped with a Heated Electrospray lonization probe (HESI-II) and operating in full MS scan mode. MS/MS data were acquired at different fragmentation energies (NCE 25, 35 and 50) on pooled samples (QC) consisting of an equimolar mixture of all the samples in the study. HILIC experiments were performed using a ZIC-HILIC column 2.1 x 100 mm, 3.5 µm, 200 Å (Merck Millipore) and mobile phase solvents consisting of 10 mM ammonium acetate in 50/50 acetonitrile/water (A) and 10 mM ammonium acetate in 95/5 acetonitrile/water (B). RPLC experiments were performed using a Hypersil GOLD column 2.1 x 150 mm, 1.9 µm, 175 Å (Thermo Scientific) and mobile phase solvents consisting of 0.06% acetic acid in water (A) and 0.06% acetic acid in methanol (B).

Data quality was ensured by: (1) sample randomization for metabolite extraction and data acquisition, (2) multiple injections of a pooled sample to equilibrate the LC-MS system prior to running the sequence (12 and 6 injections for HILIC and RPLC methods, respectively), (3) spike-in labeled IS during sample preparation to control for extraction efficiency and evaluate LC-MS performance, (4) checking mass accuracy, retention time and peak shape of the IS in each sample and (5) injection of a pooled sample every 10 injections to control for signal deviation over time.

Data processing. Data from each mode were independently analyzed using Progenesis QI software (v2.3) (Nonlinear Dynamics). Metabolic features from blanks and that did not show sufficient linearity upon dilution in QC samples (r < 0.6) were discarded. Only metabolic features present in > ⅔ of the samples were kept for further analysis. Inter- and intra-batch variations were corrected by applying locally estimated scatterplot smoothing local regression (LOESS) on pooled samples injected repetitively along the batches (span = 0.75). Data were acquired in four batches for HILIC and RPLC modes. Dilution effects were corrected using probabilistic quotient normalization (PQN) (M. E. Coussons-Read, Obstet Med. 2013; 6:52-57, the disclosure of which is incorporated herein by reference). Missing values were imputed by drawing from a random distribution of low values in the corresponding sample. Multiple aliquots (1 to 4) were analyzed for each sample (n = 172 from 99 unique samples). Data from replicates were aggregated by taking the mean (n = 2) or median (n = 3 to 4). Data from each mode were then merged, producing a dataset containing 6,630 metabolic features. Metabolite abundances were reported as spectral counts.

Metabolic feature annotation. Peak annotation was first performed by matching experimental m/z, retention time and MS/MS spectra to an in-house library of analytical-grade standards. Remaining peaks were identified by matching experimental m/z and fragmentation spectra to publicly available databases including HMDB (www.hmdb.ca/), MoNA (mona.fiehnlab.ucdavis.edu/) and MassBank (www.massbank.jp/) using the R package ‘MetID’ (v0.2.0) (L. Schiffer, et al., J Steroid Biochem Mol Biol. 2019; 194:105439, the disclosure of which is incorporated herein by reference). Briefly, metabolic feature tables from Progenesis QI were matched to fragmentation spectra with a m/z and a retention time window of ± 15 ppm and ± 30 s (HILIC) and ± 20 s (RPLC), respectively. When multiple MS/MS spectra match a single metabolic feature, all matched MS/MS spectra were used for the identification. Next, MS1 and MS2 pairs were searched against public databases and a similarity score was calculated using the forward dot-product algorithm which considers both fragments and intensities (T. T. M. Ngo, et al., Science. 2018; 360:1133-1136, the disclosure of which is incorporated herein by reference). Metabolites were reported if the similarity score was above 0.4. Spectra from metabolic features of interest important in random forest models (see below) were further investigated manually to confirm identification.

A random forest algorithm was used to build multivariate prediction models to estimate GA at the time of sample collection using all samples (n = 99), samples from term (n = 49) and samples from preterm deliveries (n = 50). The parameters of the models were optimized using internal cross-validation and an external leave-one-out cross-validation strategy was implemented to test the predictions on the excluded sample. This process was repeated 99 times and the final result was reported as an aggregate of all blinded predictions. A restricted model containing 3 metabolites was developed and validated using an independent cohort (n = 20, Stanford cohort).

Superclass level classification was performed using International Chemical Identifiers (InChl) keys for unique metabolic features (n = 2,192) using the ClassyFire Batch search cfb.fiehnlab.ucdavis.edu/ (B. Vwalika, et al., Int J Gynaecol Obstet. 2017; 136:180-187, the disclosure of which is incorporated herein by reference). The Mummichog 1 algorithm was used in the web tool MetaboAnalyst 4 to search for enriched pathways (see R. A. Carer, et al., Metabolomics. 2019; 15:124; and AMANHI (Alliance for Maternal and Newborn Health Improvement), et al., J Glob Health. 2017; 7:021202; the disclosures of which are each incorporated herein by reference). Mummichog leverages the organization of metabolic networks to predict functional activity directly from metabolic feature tables, bypassing metabolite identification. Significance of pathways was determined by the one-sided Fisher exact t-test using KEGG pathways. P-values ≤ 0.05 were considered significant. Visualization of metabolites belonging to significant pathways on the KEGG map was generated using network explorer tool in MetaboAnalyst 4.

Pairwise Spearman’s rank correlations were calculated using the R package ‘Hmisc’ (v3.15-0) and weighted, undirected networks were plotted with ‘igraph’ (v0.7.1). Correlations with Bonferroni adjusted P-values ≤ 0.01 were included and displayed via the Fruchterman-Reingold method. Nodes were color-coded by significance in the term and preterm models with node size representing the betweenness centrality.

### Results

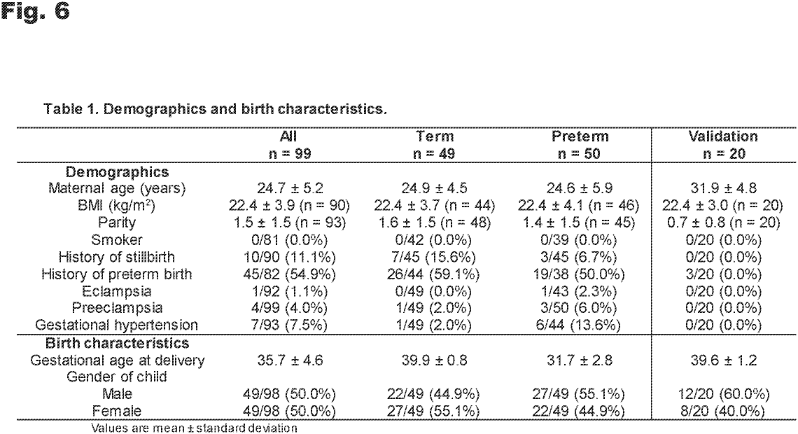

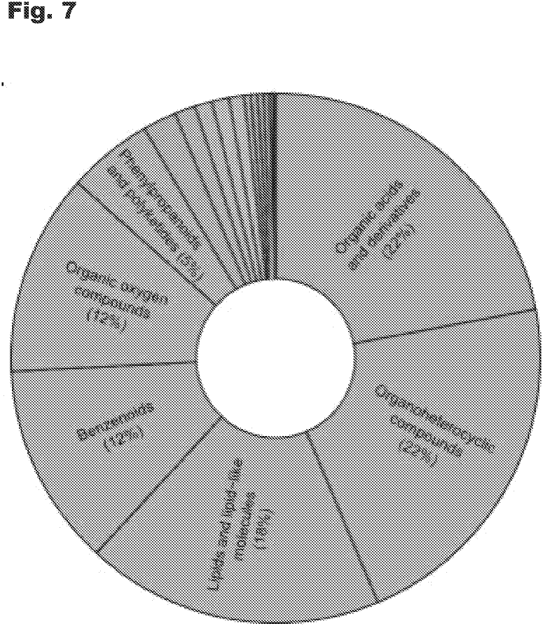

A total of 99 urine samples from term (≥37 weeks’ GA, n = 49) and preterm (<37 weeks’ GA, n = 50) pregnancies collected between 8 and 19 weeks of gestation were selected from each of the five AMANHI and GAPPS sites (FIGS. 4 & 5). Participant demographics and birth characteristics are presented in FIG. 6. Urinary metabolites were profiled using an untargeted metabolomics platform that combines hydrophilic interaction chromatography (HILIC) and reverse phase liquid chromatography (RPLC) coupled with high resolution mass spectrometry. After data curation, 6,630 metabolic features representing a wide chemical diversity were retained, including organic acids (22%), organoheterocyclic compounds (22%), lipids and lipid-like molecules (18%), benzenoids (12%), organic oxygen compounds (12%) and other minor chemical classes (FIG. 7). A large proportion (21%) of lipids and lipid-like molecules were steroid hormones, which is expected for samples collected during pregnancy.

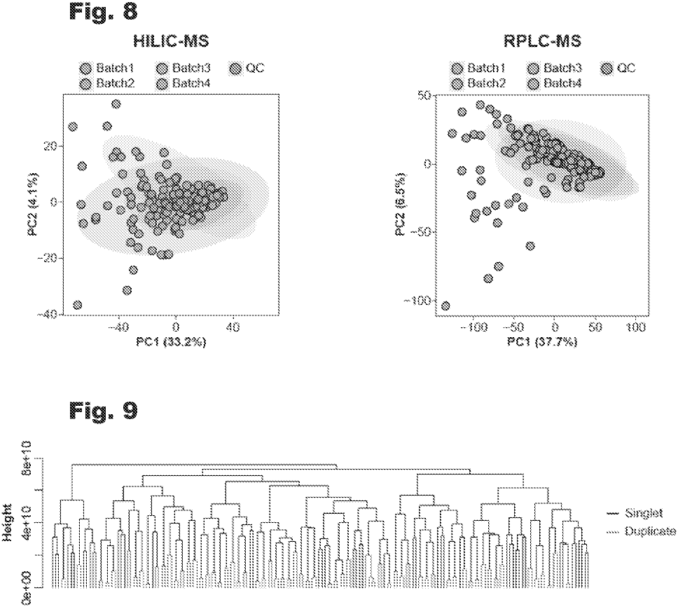

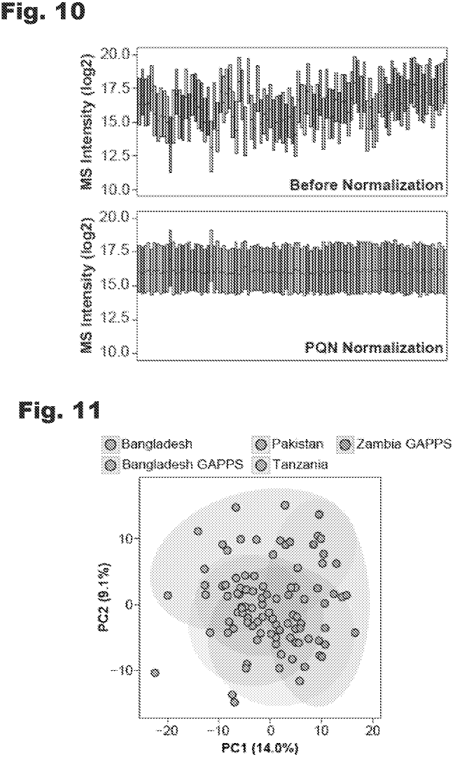

The quality of the dataset was first examined to ensure technical reproducibility and the absence of a batch effect (FIG. 8). Pooled samples (QC) clustered together and samples analyzed in different batches were highly concordant on principal component analysis (PCA) plots. In addition, replicate samples from distinct aliquots processed and analyzed in a random order (n = 172 from 99 samples) clustered together, indicating high reproducibility and robustness of the metabolomic platform (FIG. 9). Urine concentrations can vary substantially depending on the hydration state of the participant. This can be visualized in FIG. 10 with variable distribution of MS signal intensity detected in each individual. We applied probabilistic quotient normalization (PQN) that successfully eliminated the dilution effect.

A concern when collecting samples from different sites pertains to variability in metabolite levels due to differential sample collection (e.g., time of collection, fasting status, clean catch) and handling (e.g., timing of processing, freezing and transportation) procedures. This is especially true for those metabolites that are susceptible to enzymatic activity and degradation. Urine samples collected at different sites were mostly overlapping on a PCA plot, suggesting minor effects related to collection sites (FIG. 11) and validates the standard operating procedure followed by the different sites. Hence, all the samples provided for this analysis could be used together to investigate the ability of urinary metabolites to predict GA.

### Prediction of Gestational Age at Sample Collection

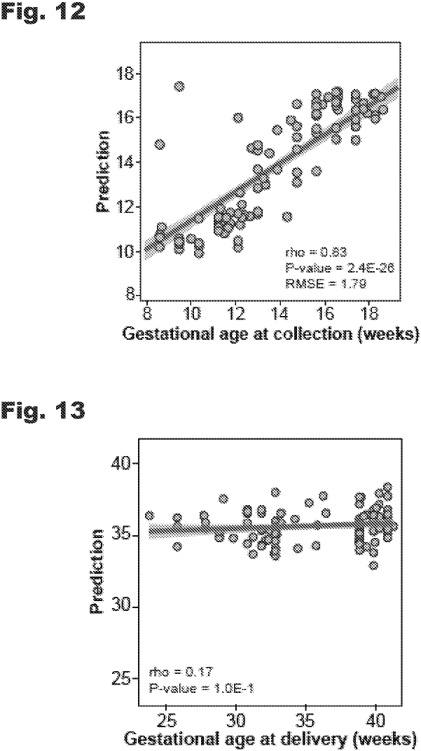

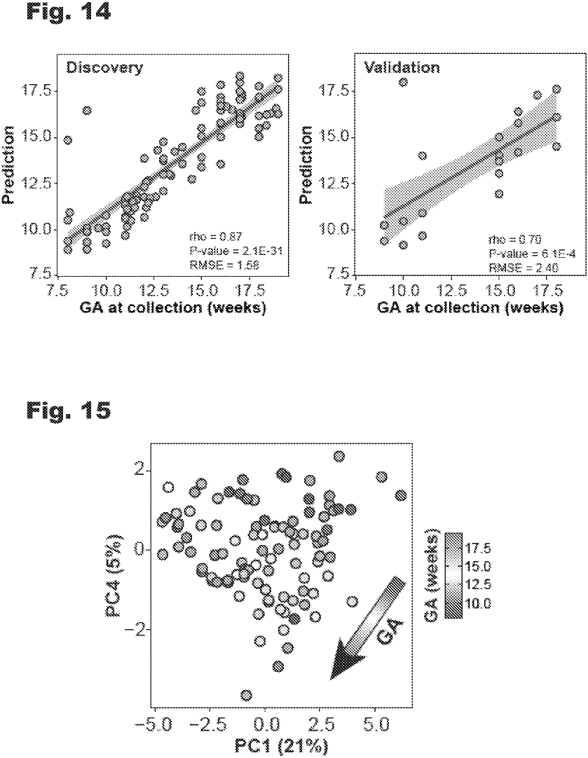

It was next investigated whether urine metabolites could be used to accurately predict GA at time of collection. A random forest (RF) algorithm was employed using all 6,630 metabolic features and yielded a model that could predict GA at collection with a cross-validated Spearman coefficient of correlation of 0.83 (P-value = 2.4E-26 and a root mean squared error (RMSE) = 1.79 weeks) (FIG. 12). However, urine metabolite levels were not successful in predicting GA at delivery (FIG. 13). For potential use in a field setting, a restricted model was generated using the least number of metabolites while retaining predictive ability. The restricted model included three metabolites and yielded excellent predictive ability (rho = 0.87, P-value = 2.1E-31 and RMSE = 1.58 weeks) (FIG. 14). This parsimonious model was validated using samples from an independent cohort of healthy pregnancies (n = 20, rho = 0.70, P-value = 6.1E-04 and RMSE = 2.40 weeks). Among three metabolites selected in the model two were uncharacterized molecules with steroid-like structures (C19H28O8S and C25H34O10) and one was an estrogen (estriol glucuronide). It should be noted that GA for two urine samples were overestimated by the model. This was explained by an overcorrection of the MS signal by the normalization procedure for these samples that were the most diluted in the study.

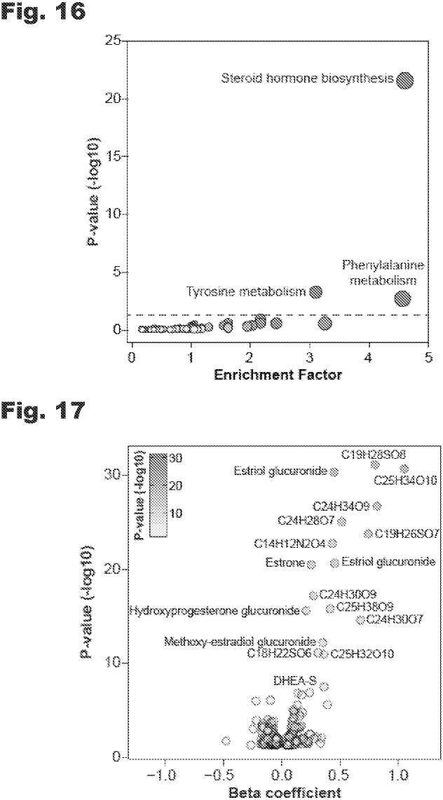

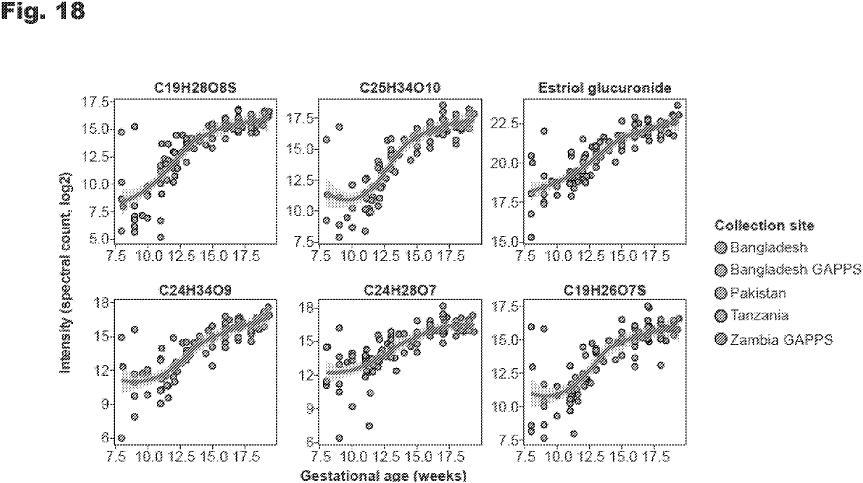

Biological processes tracking with pregnancy progression were then investigated by using significant metabolites from the original predictive model (752/6,630 with P-value < 0.05). The timing of sampling could be visualized along two dimensions by plotting the principal components (PCs) PC1 and PC4 that were most strongly associated with GA (FIG. 15). Pathway enrichment analysis was performed using the Mummichog 1 algorithm. Steroid hormone biosynthesis (P-value = 2.9E-22) was significantly associated with GA (FIG. 16) and involved a myriad of steroid hormones and their derivatives, such as estrogen derivatives (e.g. estriol glucuronide, estrone and estradiol glucuronide), progesterone derivatives (e.g. hydroxyprogesterone glucuronide, hydroxyprogesterone and progesterone), corticosteroids (e.g. tetrahydrodeoxycorticosterone [THDOC]) and androgens (e.g. dehydroepiandrosterone sulfate [DHEA-S]) (FIG. 17). As expected, all of these molecules were positively associated with GA (FIG. 18). In addition, many uncharacterized molecules with steroid-like structures were strongly associated with GA including sulfated molecules (e.g. C19H28O8S and C19H26O7S) and potential glucuronide derivatives (e.g. C25H34O10 and C24H34O9). Even though most significant metabolites were positively associated with GA (55%), a large proportion presented a negative association (45%) (FIG. 17). In addition to the steroid pathway, tyrosine (P-value = 5.9E-04) and phenylalanine metabolism (P-value = 2.2E-03) were moderately associated with GA. Metabolites belonging to significant pathways were visualized on a KEGG map.

### Differential Gestational Age Prediction in Term and Preterm Cohorts

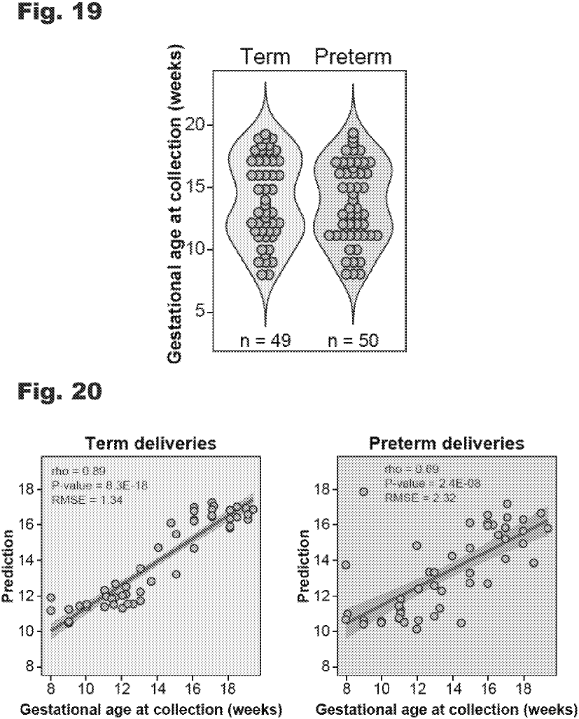

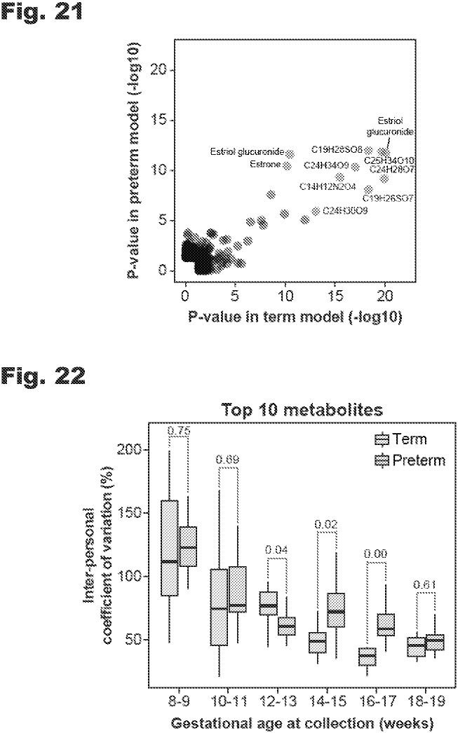

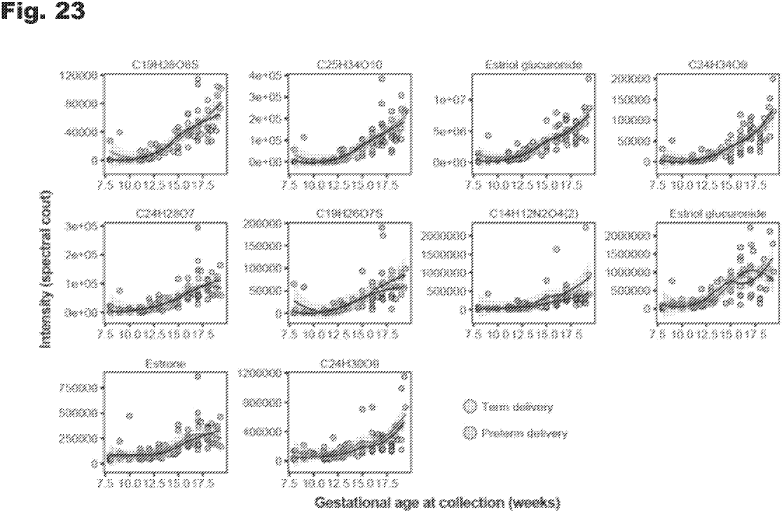

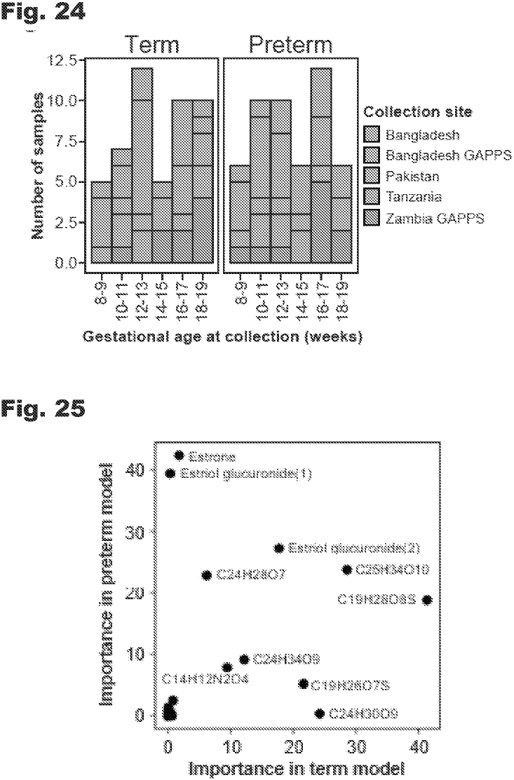

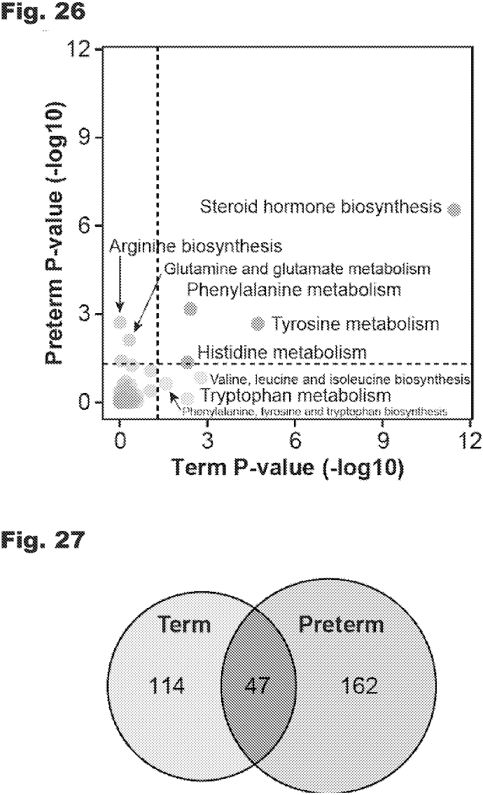

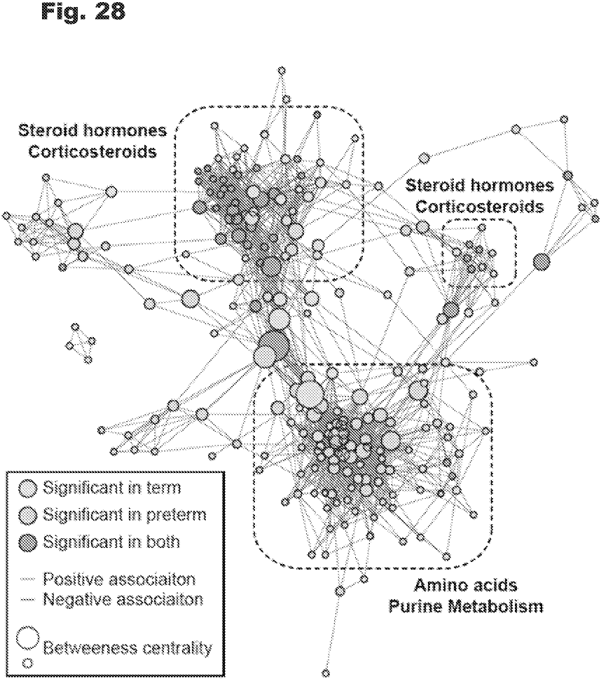

Next, it was sought to investigate GA prediction in samples collected from women who would go on to deliver at term (n = 49) versus preterm (n = 50, <37 weeks GA). GA at collection did not differ between the two groups (term: 13.60 weeks [11.50 -17.00], preterm: 13.35 [11.10 -16.52], P-value = 0.64) (FIG. 19). The RF algorithm yielded a model that performed better among the term deliveries (rho = 0.89, P-value = 8.3E-18 and RMSE = 1.34 weeks) than among the preterm deliveries (rho = 0.69, P-value = 2.4E-08 and RMSE = 2.32 weeks) (FIG. 20). Most metabolites selected in these models were also significant in the model that used all samples, with 66% and 60% overlap in term and preterm models, respectively. The metabolites driving both term and preterm models were identical however, they could predict GA better in the term cohort as indicated by their smaller P-values (FIG. 3C). This was not explained by differential metabolite trajectories or abundances but rather by a higher inter-individual variability of their absolute levels between weeks 14 and 17 (FIGS. 22-24). Even though the top metabolites selected in both models were the same, the most important metabolites differed with estrogens (estrone and estriol glucuronide) and uncharacterized metabolites (C19H26O7S and C24H30O9) being more important in the preterm and the term models, respectively (FIG. 25).

Pathway enrichment analysis confirmed the results from the general model with significant enrichment of steroid hormone biosynthesis, phenylalanine and tyrosine metabolism in both models (FIG. 26). Interestingly, certain pathways were enriched exclusively in the term and preterm models. Valine, leucine and isoleucine biosynthesis (P-value = 1.6E-03) as well as tryptophan metabolism (P-value = 3.7E-03) were associated with GA in term pregnancies, while arginine biosynthesis (P-value = 1.9E-03) and glutamine and glutamate metabolism (P-value = 7.7E-03) were associated with GA in preterm pregnancies. Correlation network analysis revealed two clusters of highly correlated metabolites (FIGS. 27 & 28). One cluster was composed of steroid hormones, with a majority of metabolites selected in both models. A second cluster was mostly composed of amino acids (9/20 amino acids including 3 branched chain amino acids as well as acetylated amino acids) and purine metabolites (purine nucleosides guanosine and inosine as well as their methylated forms), and was exclusively selected in the preterm model. These differences may reflect dysregulated biological processes associated with PTB.

### Conclusions

In this work, urinary metabolites accurately predicted GA at time of collection from samples collected in the first and early second trimesters of pregnancy from diverse geographies. The predictions were robust whether the pregnancy went to term or ended prematurely. These findings are in line with recent reports showing that maternal blood metabolites can successfully predict GA. The currently described method provides a simpler alternate for GA dating using urine, which can be collected non-invasively and requires minimal processing. This is in contrast to blood that requires specific collection, handling and processing to retain sample integrity.

This example also shows that implementing standard operating procedures for urine collection across sites is feasible without site effects by utilizing global metabolic profiling. The LC-MS approach was robust and sensitive with the detection of a wide variety of chemicals belonging to 187 “Superclass level” of the ClassyFire classification system.

Regression RF selected a set of urine metabolites that accurately predicted GA. Steroid hormones and their derivatives including estrogens, progesterones, corticosteroids and androgens were among the strongest predictors. For instance, progesterone and 17-hydroxyprogesterone were detected, which have previously been shown to be strongly associated with the length of gestation and are widely recommended for women at high risk for PTB in countries with a very high human development index. The level of THDOC, estriol glucuronide, progesterone, and DHEA-S were among the top predictors in urine reflecting recent findings in plasma. The roles of progesterone, estriol glucuronide, and DHEA-S in pregnancy are well described, however, neurosteroid THDOC has been less studied. These molecules present value to monitor the length of pregnancy and may also prove useful to detect pregnancy conditions such as prenatal stress and their impact on pregnancy outcome and long-term infant health and development.

The untargeted metabolomics platform also detected many uncharacterized molecules that were defined by their elemental composition. Interestingly, many of these molecules were associated with GA at sampling and hold a higher predictive ability than many molecules previously described in the literature. For example, 7 of the top 10 metabolites were uncharacterized with C19H28O8S and C25H34O10 being the two most predictive analytes. These molecules are likely conjugated steroids with the former containing a sulfate and the latter a glucuronic acid moiety. Conjugated molecules are abundant in urine since conjugation increases their solubility and facilitates urinary excretion. These results highlight the value of untargeted LC-MS metabolomics approaches for the sensitive and simultaneous profiling of many steroid metabolites and derivatives giving insights into steroid biosynthesis and excretion processes.

A restricted model was constructed that uses the abundance of only three metabolites and show that it can estimate the GA early in pregnancy with better accuracy (RMSE = 1.6 weeks) than models developed in blood using cell-free RNA (RMSE = 4.3 weeks) (S. E. Stein and D. R. Scott, J Am Soc Mass Spectrom. 1994; 5:859-66, the disclosure of which is incorporated herein by reference) or metabolites (RMSE = 2.5 weeks) (L. Liang, et al., Cell. 2020; 181:1680-92, the discosure of which is incorporated herein by reference). Importantly, the restricted model was generalizable when applied to an independent cohort of healthy pregnancies. Additional work with larger sample sets will likely improve model performance. In contrast to recent studies that identified molecular signatures associated with GA using multiple samples per pregnancy collected at a single site, urinary metabolites can accurately estimate GA as compared to ultrasound dating using a single time point per pregnancy from populations across multiple countries. With the objective of developing a clinical test, the two unknown metabolites C19H28O8S and C25H34O10 will need to be fully characterized and it remains to be determined if the model performs well on samples collected before week 8 and after week 19.

Regression RF prediction models were also generated to predict GA in samples from mothers that delivered term and preterm (<37 weeks GA). Even though the same metabolites (i.e. steroid hormones) were the most predictive in both models, the prediction performance was higher for term deliveries. This observation may in part reflect a tighter control of the level of these molecules in term pregnancies rather than a difference in their absolute abundance. Correlation network analysis revealed a cluster of amino acids and purine metabolites mainly selected in the preterm model encompassing differences in these pathways in term and preterm pregnancies. Many of these molecules have been reported as being dysregulated in PTB including choline, dimethylarginine, methionine, phenylalanine, tryptophan, valine, threonine, isoleucine, leucine and xanthine. Targeted and untargeted metabolomics approaches have been employed to study PTB and have identified various early biomarkers. However, very little consensus has yet emerged owing to varying maternal sample sources (i.e. cervicovaginal fluid, amniotic fluid, blood and urine), GA at sampling and participant demographics.

In conclusion, this study demonstrated that a small set of urinary metabolites can predict GA using a single sample in a diverse cohort. This approach can benefit pregnant women worldwide because collection of urine is non-invasive and it does not require processing.

### DOCTRINE OF EQUIVALENTS

While the above description contains many specific embodiments of the invention, these should not be construed as limitations on the scope of the invention, but rather as an example of one embodiment thereof. Accordingly, the scope of the invention should be determined not by the embodiments illustrated, but by the appended claims and their equivalents.

What is claimed is:

1. A method for determining gestational age of a pregnant individual, the method comprising:
measuring one or more analytes of a urine sample collected from an individual; and
estimating, using a predictive computational model and the one or more analyte measurements, a gestational age of the individual.

2. The method of claim 1 further comprising:
collecting the urine sample from the individual.

3. The method of claim 1, wherein the urine sample is collected before 20 weeks of gestation.

4. The method of claim 1, wherein the urine sample is collected between 8 and 19 weeks of gestation.

5. The method of claim 1, wherein a single urine sample is utilized to determine gestational age.

6. The method of claim 1, wherein two or more urine samples are collected from the individual, and wherein the gestational age is estimated using the predictive computational model and analyte measurements from each of the two or more urine samples.

7. The method of claim 1, wherein the predictive computational model is one of: ridge regression, K-nearest neighbors, LASSO regression, elastic net, least angle regression (LAR), random forest linear regression, or principal components analysis.

8. The method of claim 1, wherein the one or more of the analyte measurements comprises a measurement of one of the following analytes: C19H28O8S, C25H34O10, or estriol glucuronide.

9. The method of claim 1, wherein the one or more of the analyte measurements comprises a measurement of two of the following analytes: C19H28O8S, C25H34O10, or estriol glucuronide.

10. The method of claim 1, wherein the one or more of the analyte measurements comprises a measurement of the following analytes: C19H28O8S, C25H34O10, and estriol glucuronide.

11. The method of claim 1, wherein the one or more of the analyte measurements comprises a measurement of one of the following analytes: estriol glucuronide, C19H28O8S, C25H34O10, C24H28O7, C24H34O9, C19H26SO7, C14H12N2O4, C24H30O9, or estrone.

12. The method of claim 1, wherein the one or more of the analyte measurements comprises a measurement of two of the following analytes: estriol glucuronide, C19H28O8S, C25H34O10, C24H28O7, C24H34O9, C19H26SO7, C14H12N2O4, C24H30O9, or estrone.

13. The method of claim 1, wherein the one or more of the analyte measurements comprises a measurement of three of the following analytes: estriol glucuronide, C19H28O8S, C25H34O10, C24H28O7, C24H34O9, C19H26SO7, C14H12N2O4, C24H30O9, or estrone.

14. The method of claim 1, wherein the one or more of the analyte measurements comprises a measurement of four of the following analytes: estriol glucuronide, C19H28O8S, C25H34O10, C24H28O7, C24H34O9, C19H26SO7, C14H12N2O4, C24H30O9, or estrone.

15. The method of claim 1, wherein the one or more of the analyte measurements comprises a measurement of five of the following analytes: estriol glucuronide, C19H28O8S, C25H34O10, C24H28O7, C24H34O9, C19H26SO7, C14H12N2O4, C24H30O9, or estrone.

16. The method of claim 1, wherein the one or more of the analyte measurements comprises a measurement of the following analytes: estriol glucuronide, C19H28O8S, C25H34O10, C24H28O7, C24H34O9, C19H26SO7, C14H12N2O4, C24H30O9, and estrone.

17. The method of claim 1, wherein the individual has been diagnosed as pregnant.

18. The method, wherein the individual has not been diagnosed as pregnant.

19. The method of claim 1 further comprising performing sonography on the individual.

20. The method of claim 1 further comprising:
treating the individual based on the estimated gestational age, wherein the treatment is one of: medication, dietary supplement, Caesarian delivery, or surgical procedure.
